(19) United States
(12) Patent Application Publication
(10) Pub. No.: US 2011/0152980 A1
(43) Pub. Date: Jun. 23, 2011

(54) METHOD AND APPARATUS FOR ACTIVATING PHYSIOLOGICAL FUNCTIONS

(76) Inventors: Takehito Saito, Kusatsu-shi (JP); Yuko Hara, Kusatsu-shi (JP); Jun-Ichiro Arai, Kusatsu-shi (JP); Kenkichi Kagawa, Sakai-shi (JP)

(21) Appl. No.: 12/672,241

(22) PCT Filed: Aug. 5, 2008
(86) PCT No.: PCT/JP2008/064053

§ 371 Date: Feb. 4, 2010

(30) Foreign Application Priority Data
Aug. 7, 2007 (JP) 2007-204834

(51) Int. Cl. A61F 7/00

(52) U.S. Cl. 607/96

(57) ABSTRACT

With a method and an apparatus for activating physiological functions, a thermal stimulus at 31° C. to 34° C. and a cold stimulus at 18° C. to 22° C. are alternately given to the human body to activate the human physiological functions, in particular, the motor functions of peripheral blood vessels. As a result, the blood pressure regulatory functions, the blood circulation regulatory functions, and the thermoregulatory functions are enhanced thereby to achieve the effects of preventing hypertension, preventing reduction in metabolism and repair functions, accelerating recovery from fatigue, and preventing heat strokes. The apparatus for activating physiological functions includes, for example, a heat pump type air conditioner that selectively generates hot air and cool air. The hot air blown out from the air conditioner into a room interior is utilized as the thermal stimulus, and the cool air also blown out from the air conditioner is utilized as the cold stimulus.

### TECHNICAL FIELD

The present invention relates to a method for activating human physiological functions such as the motor functions of peripheral blood vessels, and an apparatus therefor.

### BACKGROUND ART

Of human physiological functions, the motor functions of peripheral blood vessels, in particular, play an important role in the regulation of blood pressure, blood circulation, and body temperature. A decrease in the motor functions of peripheral blood vessels leads to various physical disorders, causing a problem in leading a healthy life. The physical disorders caused by a decrease in the motor functions of peripheral blood vessels include, for example, hypertension as a result of impaired blood pressure regulatory functions, delayed recovery from fatigue because of reduced metabolism and repair functions as a result of impaired blood circulation regulatory functions, and proneness to heat strokes as a result of impaired thermoregulatory functions.

Patent Documents 1 and 2 disclose air conditioners that measure the physiological conditions of the air conditioner user and control the operation state in accordance with the measured physiological conditions. As the indices representing the physiological conditions of the air conditioner user, the air conditioner disclosed in Patent Document 1 uses, for example, the skin temperature, heart beat, and electro-dermal activity of the air conditioner user, while the air conditioner disclosed in Patent Document 2 uses, for example, the skin temperature or skin perspiration rate of the air conditioner user. However, neither of the air conditioners in Patent Documents 1 and 2 activates the human physiological functions. For example, although a person might be prevented from having a poor circulation in a room air-conditioned by the air conditioner of Patent Document 1 or 2, it does not mean that this same person is prevented from having a poor circulation somewhere else other than inside of this air-conditioned room.

Patent Document 3 discloses a hot air heater that generates hot air with its temperature or air volume being changed temporally irregularly. This hot air heater aims at stimulating the autonomic nerves of the heater user thereby to enhance the relaxed conditions of the heater user. However, this hot air heater is not much expected to be effective in respect of activating the human physiological functions, in particular of enhancing the motor functions of peripheral blood vessels.
Patent Document 1: Japanese Laid-Open Patent Publication No. 2004-125376
Patent Document 2: Japanese Laid-Open Patent Publication No. 2003-42508
Patent Document 3: Japanese Laid-Open Patent Publication No. 2001-141306

### DISCLOSURE OF THE INVENTION

Accordingly, an objective of the present invention is to provide a method and an apparatus for activating human physiological functions, in particular by enhancing the motor functions of peripheral blood vessels, thereby to achieve the effects of, for example, preventing hypertension, preventing reduction of metabolism and repair functions, accelerating recovery from fatigue, and preventing heat strokes.

The inventors of the present application have verified through the following experiment that the human physiological functions are activated, in particular the motor functions of peripheral blood vessels are enhanced, by alternately giving to the human body a thermal stimulus and a cold stimulus.

A testing room installed with a heat pump type air conditioner that selectively generates hot air and cool air was prepared (see FIG. 1), and the test subject was placed in this room. As shown in FIG. 11, the temperature of air blown out from the air conditioner was gradually increased taking 25 minutes so that the room temperature changed from 20° C. (or 18° C. to 22° C.) to 32° C. (or 31° C. to 34° C.), after which the temperature was held at 32° C. for 10 minutes. After that, the temperature of the blown-out air was gradually decreased taking 25 minutes so that the room temperature changed from 32° C. to 20° C. The same person being tested was subjected to the temperature changes in such a pattern for 1 hour or more per day for 5 consecutive days.

FIG. 12 shows measurement results of the reactivity of the test subject's fingertip skin temperature in response to cold water before and after such an experiment. As shown in FIG. 12, when the fingertips were dipped in cold water, whether before or after the experiment, the fingertip skin temperature decreased rapidly. However, it was ascertained that, the fingertip skin temperature recovered earlier after the experiment as compared to before the experiment. This can be interpreted as indicating that alternately giving to a human body a thermal stimulus and a cold stimulus enhances the reactivity of the fingertip skin temperature, i.e., enhances the motor functions of the peripheral blood vessels.

Based on the above verified fact, to achieve the above-noted objective, one aspect of the present invention provides a method for activating physiological functions, wherein a thermal stimulus in a first temperature range of from 31° C. to 34° C. and a cold stimulus in a second temperature range of from 18° C. to 22° C. are alternately given to a human body.

The temperature changed from the first temperature range to the second temperature range may continuously be maintained for a predetermined time within the second temperature range. The temperature changed from the second temperature range to the first temperature range may continuously be maintained for a predetermined time within the first temperature range. In this case, the thermal stimulus and/or cold stimulus given to the human body is/are increased, as a result of which the effect of activating the human physiological functions, in particular of enhancing the motor functions of peripheral blood vessels, is more reliably achieved.

The thermal stimulus and the cold stimulus are preferably given to the human body alternately for 1 hour or more per day for five or more consecutive days. In this case, the effect of activating the human physiological functions, in particular of enhancing the motor functions of peripheral blood vessels, achieved by alternately giving to the human body the thermal stimulus and the cold stimulus is stably maintained for a long period of time.

Another aspect of the present invention provides an apparatus for activating physiological functions, including temperature changing means configured to give alternately to a human body a thermal stimulus in a first temperature range of from 31° C. to 34° C. and a cold stimulus in a second temperature range of from 18° C. to 22° C.

The temperature changing means may be a heat pump type air conditioner that selectively generates hot air and cool air. Alternatively, the temperature changing means may include a cold heat generating unit having a Peltier element or a cooling machine and a heat generating unit having a Peltier element or a heater. In this case, the apparatus for activating physiological functions can be provided at a low cost.

### BRIEF DESCRIPTION OF THE DRAWINGS

FIG. 1 is a diagram illustrating the apparatus for activating physiological functions according to a first embodiment of the present invention in use;

FIG. 2 is a control block diagram of the apparatus for activating physiological functions according to the first embodiment;

FIG. 3 is a control flowchart of the apparatus for activating physiological functions according to the first embodiment;

FIG. 4 is a diagram illustrating the apparatus for activating physiological functions according to a second embodiment of the present invention in use;

FIG. 5 is a control block diagram of the apparatus for activating physiological functions according to the second embodiment;

FIG. 6 is a control flowchart of the apparatus for activating physiological functions according to the second embodiment;

FIG. 7 is a graph showing a first temperature change pattern;

FIG. 8 is a graph showing a second temperature change pattern;

FIG. 9 is a graph showing a third temperature change pattern;

FIG. 10 is a graph showing a fourth temperature change pattern;

FIG. 11 is a graph showing a temperature change pattern used to verify activation of the motor functions of blood vessels; and

FIG. 12 is a graph showing the changes with time of fingertip skin temperature that indicate the verified results regarding the activation of the motor functions of blood vessels.

### BEST MODE FOR CARRYING OUT THE INVENTION

Hereinafter, first and second embodiments of the present invention will be described.

### First Embodiment

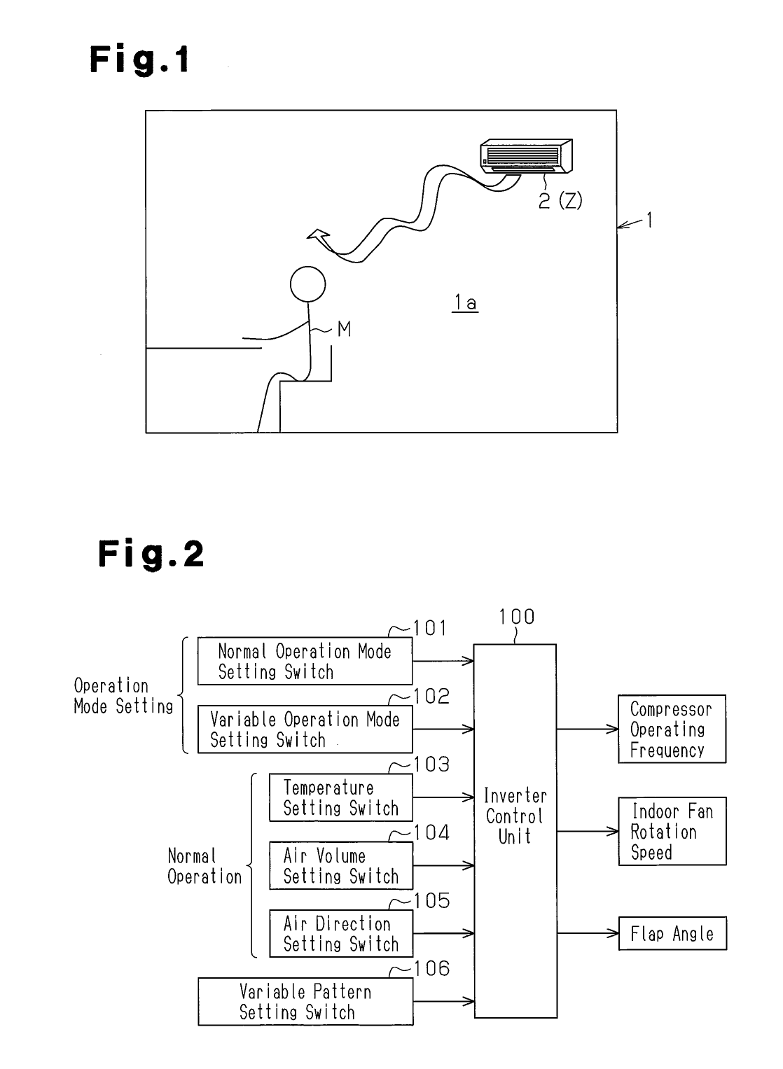

FIG. 1 shows an apparatus for activating physiological functions according to the first embodiment of the present invention. In this apparatus for activating physiological functions, a heat pump type indoor air conditioner 2 installed in a wall surface of the interior 1a of a room 1 is used as temperature changing means (temperature changing device) Z. More specifically, the hot air blown out from the air conditioner 2 into the room interior 1a during the heating operation is utilized as a thermal stimulus, while the cool air blown out from the air conditioner 2 during the cooling operation is utilized as a cold stimulus.

FIG. 2 shows a control block diagram of the air conditioner 2. The air conditioner 2 includes an inverter control unit 100. The air conditioner 2 operates in either one of two operation modes, a normal operation mode and a variable operation mode. That is, the air conditioner 2 is selectively switched over between the normal operation mode and the variable operation mode.

The normal operation mode is an operation mode in which normal cooling operation or normal heating operation is performed. Pressing a normal operation mode setting switch 101 sets the operation mode of the air conditioner 2 to the normal operation mode. The setting of air-conditioning conditions in the normal operation mode is achieved by pressing a temperature setting switch 103, an air volume setting switch 104, and an air direction setting switch 105. The inverter control unit 100 outputs control signals in accordance with the set temperature, air volume, and air direction to control the compressor operating frequency, indoor fan rotation speed, and flap angle.

On the other hand, the variable operation mode is an operation mode in which the cooling operation and heating operation are repeated alternately to cause the room temperature to fluctuate in accordance with a predetermined temperature change pattern. Such changes in the room temperature give a thermal stimulus and a cold stimulus alternately to the body of a human M in the room interior 1a, thereby activating the physiological functions of the human M, in particular the motor functions of peripheral blood vessels. As a result, the blood pressure regulatory functions, the blood circulation regulatory functions, and the thermoregulatory functions are enhanced, thereby to achieve the effects of preventing hypertension, preventing reduction of metabolism and repair functions, accelerating recovery from fatigue, and preventing heat strokes.

The variable operation mode is set as an operation mode of the air conditioner 2 by pressing the variable operation mode setting switch 102. When the air conditioner 2 is operating in the variable operation mode, pressing a variable pattern setting switch 106 can set one temperature change pattern, which is selected from several such patterns. The inverter control unit 100 outputs control signals in accordance with the set temperature change pattern to control the operation of the air conditioner 2.

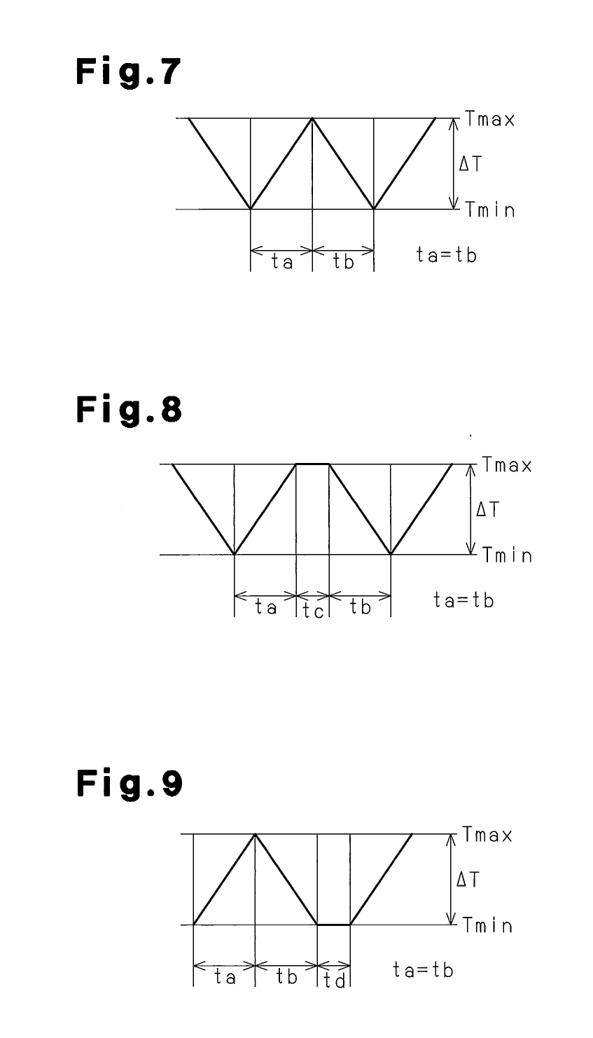

The temperature change patterns set during the variable operation mode will be described next. With the air conditioner 2 of this embodiment, one of the four basic temperature change patterns shown in FIGS. 7 to 10 is selected and set.

With the first temperature change pattern shown in FIG. 7, the room temperature changes alternately between a high temperature-side target temperature Tmax within the range of from 31 to 34° C., preferably at 32° C., and a low temperature-side target temperature Tmin within the range of from 18 to 22° C., preferably at 20° C. In this case, the amplitude of room temperature fluctuations ΔT ranges from 9 to 16° C., preferably 12° C. The air conditioner 2 performs heating operation when changing the room temperature from the low temperature-side target temperature Tmin to the high temperature-side target temperature Tmax, while it performs cooling operation when changing the room temperature from the high temperature-side target temperature Tmax to the low temperature-side target temperature Tmin. That is, the air conditioner 2 performs the heating operation and the cooling operation alternately. The hot air blown out from the air conditioner 2 into the room interior 1a during the heating operation is utilized as a thermal stimulus, while the cool air blown out from the air conditioner 2 during the cooling operation is utilized as a cold stimulus. The temperature increasing time to and the temperature decreasing time tb can be set arbitrarily, but it is preferable that they are within the range of 25 to 30 minutes and the same to each other.

The second temperature change pattern shown in FIG. 8 is different from the first temperature change pattern shown in FIG. 7 in that, after increasing the room temperature from the low temperature-side target temperature Tmin to the high temperature-side target temperature Tmax, the room temperature is maintained for a predetermined hold time tc at the high temperature-side target temperature Tmax. With the second temperature change pattern, also, the high temperature-side target temperature Tmax is preferably 32° C., the low temperature-side target temperature Tmin is preferably 20° C., and the temperature fluctuation amplitude ΔT is preferably 12° C. The temperature increasing time ta and the temperature decreasing time tb are preferably within the range of 25 to 30 minutes and the same to each other, and the hold time tc is preferably 15 minutes.

The third temperature change pattern shown in FIG. 9 is different from the first temperature change pattern shown in FIG. 7 in that, after decreasing the room temperature from the high temperature-side target temperature Tmax to the low temperature-side target temperature Tmin, the room temperature is maintained for a predetermined hold time td at the low temperature-side target temperature Tmin. With the third temperature change pattern, also, the high temperature-side target temperature Tmax is preferably 32° C., the low temperature-side target temperature Tmin is preferably 20° C., and the temperature fluctuation amplitude ΔT is preferably 12° C. The temperature increasing time ta and the temperature decreasing time tb are preferably within the range of 25 to 30 minutes and the same to each other, and the hold time td is preferably 15 minutes.

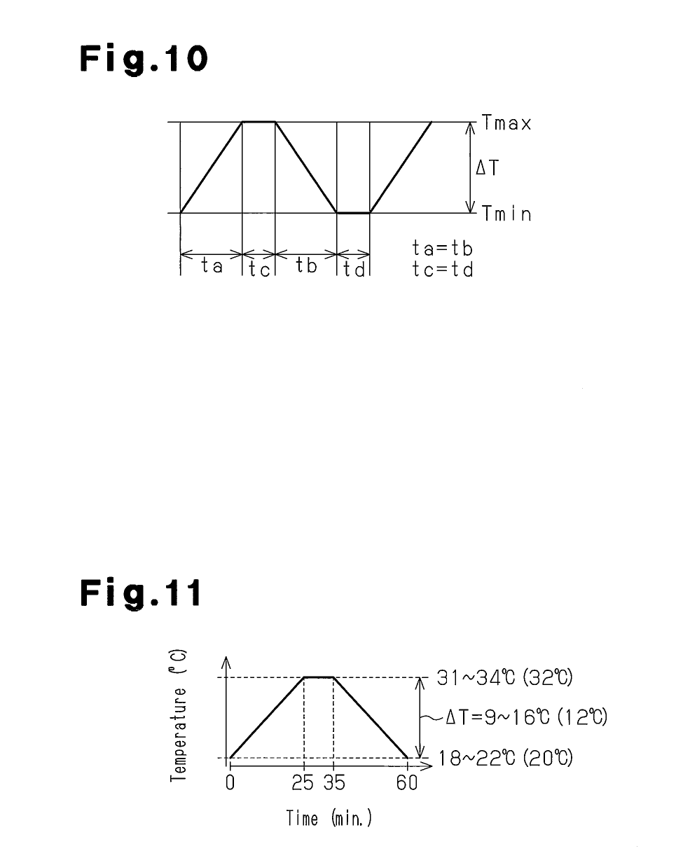

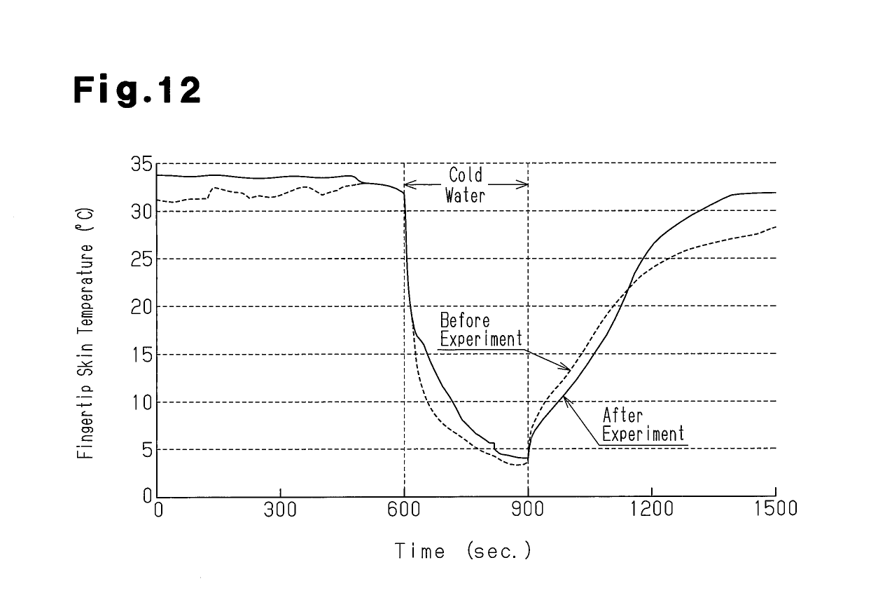

The fourth temperature change pattern shown in FIG. 10 is different from the first temperature change pattern shown in FIG. 7 in that, after increasing the room temperature from the low temperature-side target temperature Tmin to the high temperature-side target temperature Tmax, the room temperature is maintained for a predetermined hold time tc at the high temperature-side target temperature Tmax, and in that, after decreasing the room temperature from the high temperature-side target temperature Tmax to the low temperature-side target temperature Tmin, the room temperature is maintained for a predetermined hold time td at the low temperature-side target temperature Tmin. With the fourth temperature change pattern, also, the high temperature-side target temperature Tmax is preferably 32° C., the low temperature-side target temperature Tmin is preferably 20° C., and the temperature fluctuation amplitude ΔT is preferably 12° C. The temperature increasing time ta and the temperature decreasing time tb is preferably within the range of 25 to 30 minutes and the same to each other, and the high-temperature hold time tc and the low-temperature hold time td is preferably 15 minutes and the same to each other.

The temperature change pattern set during the variable operation mode is not limited to any of the four temperature change patterns shown in FIGS. 7 to 10. Naturally, the high temperature-side target temperature Tmax may be changed within the range of from 31 to 34° C., and the low temperature-side target temperature Tmin may be changed within the range of from 18 to 22° C. Alternatively, some of the four temperature change patterns shown in FIGS. 7 to 10 may be combined as desired. Alternatively, the temperature increasing time ta and the temperature decreasing time tb may be set differently from each other, or the high-temperature hold time tc and the low-temperature hold time td may be set differently from each other.

It goes without saying that the high temperature-side target temperature Tmax and the low temperature-side target temperature Tmin are merely target values, and there may be some discrepancy between the actual room temperature and these target temperatures.

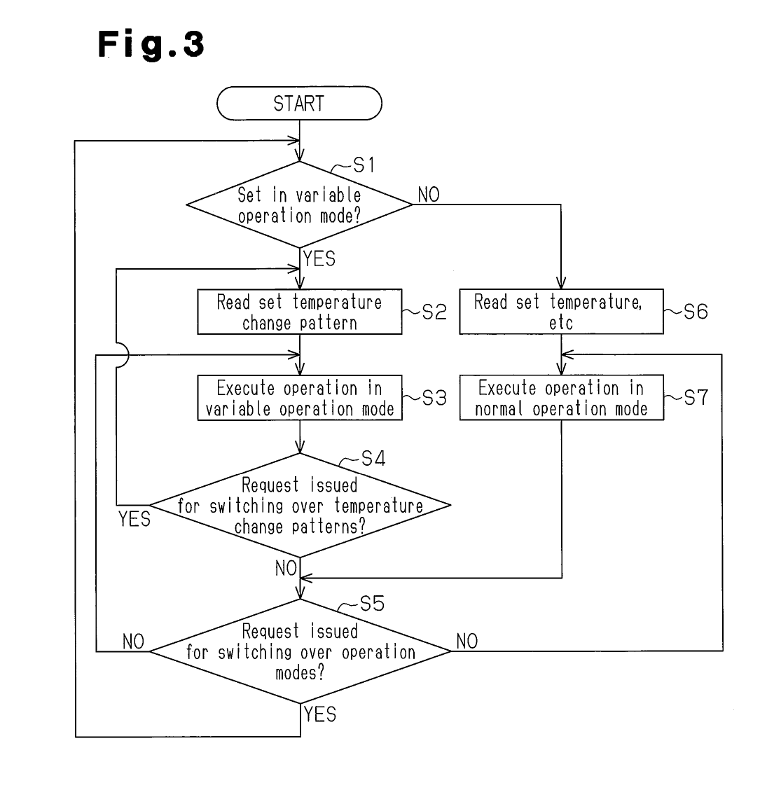

Next, modes of operation control of the apparatus for activating physiological functions according to this embodiment will be described with reference to the control flowchart of FIG. 3.

First, at step S1, the control unit 100 determines the current operation mode of the air conditioner 2. If it is determined that the air conditioner is set in the normal operation mode, the control unit 100 reads the temperature, air volume, and air direction that have been set using the setting switches 103 to 105, at step S6. At step S7, the control unit 100 causes the air conditioner 2 to perform the normal cooling operation or heating operation based on these set temperature, air volume, and air direction. The air conditioner 2 keeps operating in the normal operation mode until a request is issued to switch over the operation modes at step S5.

If, on the other hand, it is determined that the air conditioner is set in the variable operation mode at step S1, the control unit 100, at step S2, reads the temperature change pattern that has been set. At step S3, the control unit 100 causes the air conditioner 2 to perform the operation in the variable operation mode based on the temperature change pattern that has been set. That is, the air conditioner 2 performs the heating operation and the cooling operation alternately and repeatedly so as to change the room temperature alternately between the high temperature-side target temperature Tmax and the low temperature-side target temperature Tmin based on the set temperature change pattern. After that, if a request is issued to switch over the temperature change patterns at step S4, the process goes back to step S2, where the control unit 100 reads a new temperature change pattern. The air conditioner 2 keeps operating in the variable operation mode until a request is issued to switch over the operation modes at step S5.

As the air conditioner 2 operates in the variable operation mode, the human M in the room interior 1a is alternately subjected to a thermal stimulus by the hot air blown out from the air conditioner 2 and a cold stimulus by the cool air blown out also from the air conditioner 2. Placing the human M in the room 1 where the air conditioner 2 is operating in the variable operation mode for 1 hour or more per day preferably for five or more consecutive days activates the physiological functions of the human M, in particular the motor functions of peripheral blood vessels. As a result, the blood pressure regulatory functions, the blood circulation regulatory functions, and the thermoregulatory functions are enhanced, thereby to achieve the effects of preventing hypertension, preventing reduction of metabolism and repair functions, accelerating recovery from fatigue, and preventing heat strokes.

The temperature range of 31 to 34° C. used as the high temperature-side target temperature is the lower limit temperature range at which heat is expected to dissipate from the body of a human M due to perspiration, while the temperature range of 18 to 22° C. used as the low temperature-side target temperature is the lower limit temperature range at which shivering caused by coldness is not expected to occur in the body of a human M. Therefore, by setting the high temperature-side target temperature within the range of 31 to 34° C. and the low temperature-side target temperature within the range of 18 to 22° C., the activation of physiological functions of a human M by giving the body of the human M a thermal stimulus and a cold stimulus alternately can be favorably achieved.

### Second Embodiment

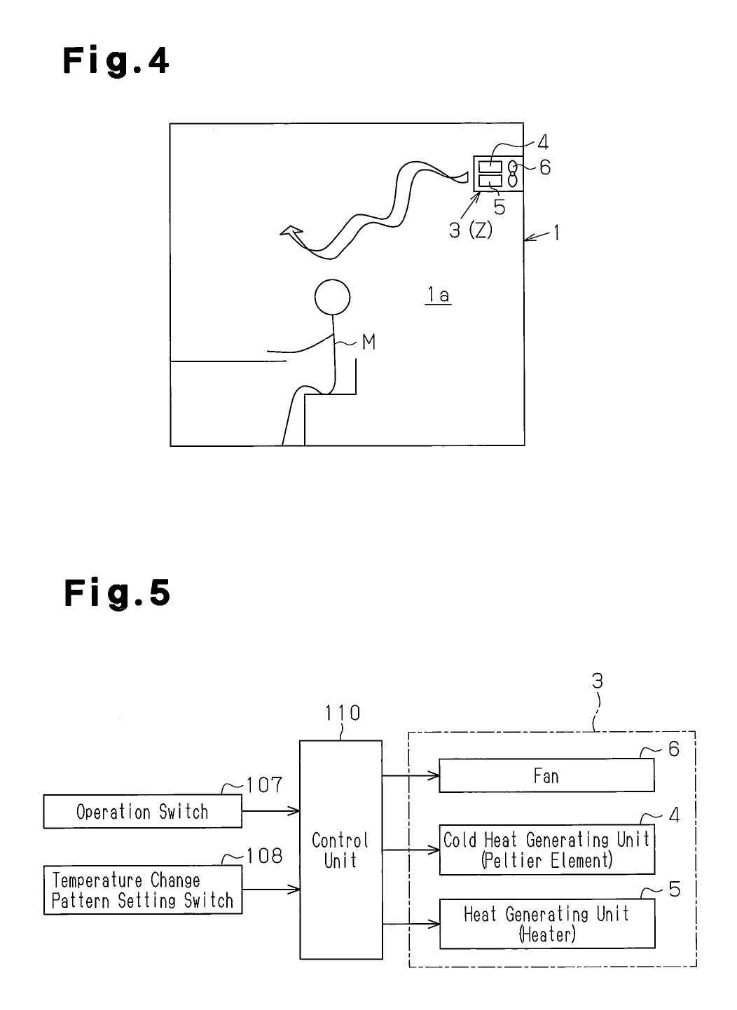

FIG. 4 shows an apparatus for activating physiological functions according to a second embodiment of the present invention. In this apparatus for activating physiological functions according to the second embodiment, a fan coil unit 3 installed in a wall surface of the interior 1a of a room 1 is used as temperature changing means (temperature changing device) Z. More specifically, the hot air blown out from the fan coil unit 3 into the room interior 1a is utilized as a thermal stimulus, while the cool air blown out from the fan coil unit 3 is utilized as a cold stimulus.

As shown in FIGS. 4 and 5, the fan coil unit 3 includes a cold heat generating unit 4 having a Peltier element (or a cooling machine), a heat generating unit 5 having a heater (or a Peltier element), and a blower fan 6. When the fan 6 is operated with power being applied to the cold heat generating unit 4, cool air is blown out from the fan coil unit 3 into the room interior 1a. On the other hand, when the fan 6 is operated with power being applied to the heat generating unit 5, hot air is blown out from the fan coil unit 3 into the room interior 1a. Blowing out cool air and hot air alternately from the fan coil unit 3 gives a cold stimulus by the cool air and a thermal stimulus by the hot air alternately to the body of a human M in the room interior 1a. In this case, the physiological functions, in particular the motor functions of peripheral blood vessels, of the human M are activated, as a result of which the blood pressure regulatory functions, the blood circulation regulatory functions, and the thermoregulatory functions are enhanced, thereby to achieve the effects of preventing hypertension, preventing reduction of metabolism and repair functions, accelerating recovery from fatigue, and preventing heat strokes.

The changes in the room temperature attained by blowing out cool air and hot air alternately from the fan coil unit 3 may be executed in accordance with the pattern selected from the temperature change patterns described in the foregoing and shown in FIGS. 7 to 10.

FIG. 5 shows a control block diagram of the apparatus for activating physiological functions according to the second embodiment. A control unit 110 receives a signal from an operation switch 107 and a signal from a temperature change pattern setting switch 108. The signal from the operation switch 107 is output to the control unit 110 in response to the operation of the operation switch 107 by the user of the fan coil unit 3. The signal from the temperature change pattern setting switch 108 is output to the control unit 110 in response to the operation of the temperature change pattern setting switch 108 by the user of the fan coil unit 3.

In response to the signal from the operation switch 107 and the signal from the temperature change pattern setting switch 108, the control unit 110 outputs control signals respectively to the cold heat generating unit 4, heat generating unit 5, and fan 6, so as to change the room temperature in accordance with the predetermined temperature change pattern. Accordingly, the cold heat generating unit 4 and the heat generating unit 5 are alternately operated, and cool air and hot air are alternately blown out by the fan 6 into the room interior 1a.

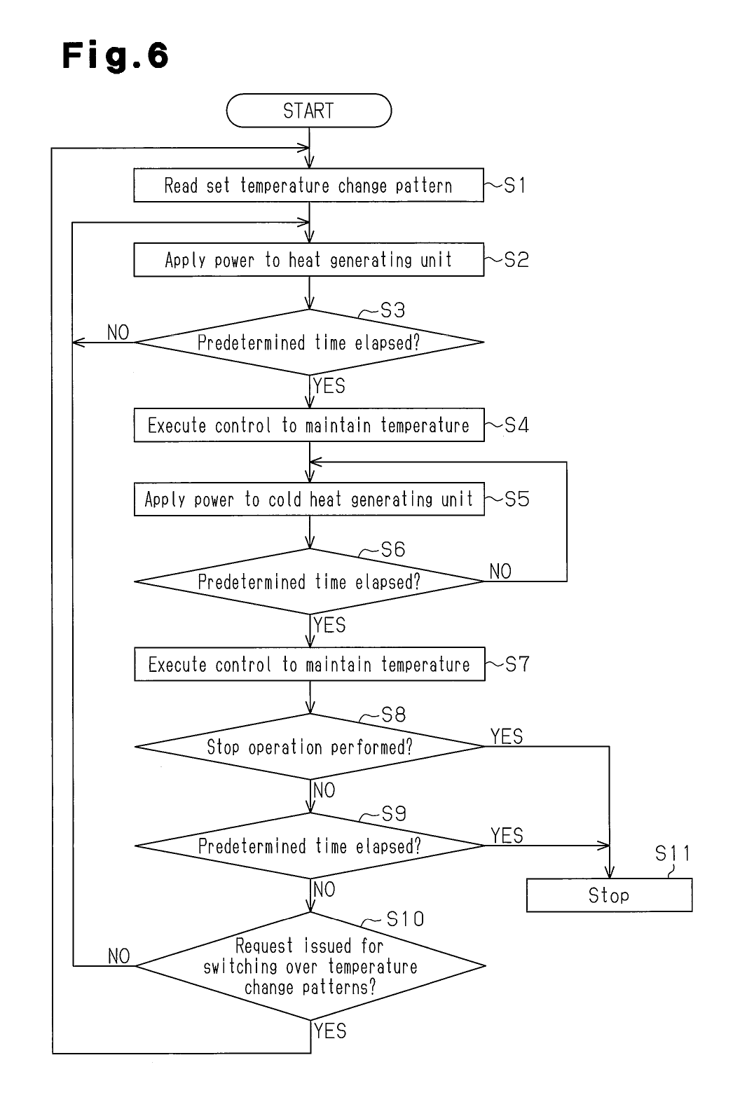

Next, modes of operation control of the apparatus for activating physiological functions according to the second embodiment will be described with reference to the control flowchart in FIG. 6.

When the operation switch 107 is operated, the control unit 110, at step S1, reads the temperature change pattern that has been set using the temperature change pattern setting switch 108. Hereinafter the description will be carried on with respect to one example wherein the temperature change pattern shown in FIG. 10 has been set.

When reading the temperature change pattern is complete, at step S2, the control unit 110 applies power to the heater of the heat generating unit 5 so as to increase the room temperature up to the high temperature-side target temperature Tmax. If, at step S3, it is determined that a predetermined time corresponding to the temperature increasing time to in FIG. 10 has elapsed, the control unit 110, at step S4, applies power to the heater of the heat generating unit 5 for a predetermined time corresponding to the high temperature hold time tc in FIG. 10, so as to maintain the room temperature at the high temperature-side target temperature Tmax.

At step S5 after that, the control unit 110 applies power to the Peltier element of the cold heat generating unit 4 in place of the heater of the heat generating unit 5 so as to decrease the room temperature down to the low temperature-side target temperature Tmin. If, at step S6, it is determined that a predetermined time corresponding to the temperature decreasing time tb in FIG. 10 has elapsed since the start of power application to the Peltier element, the control unit 110, at step S7, applies power to the Peltier element of the cold heat generating unit 4 for a predetermined time corresponding to the low temperature hold time td in FIG. 10, so as to maintain the room temperature at the low temperature-side target temperature Tmin.

At step S8 after that, if it is determined that stop operation has been performed, or, at step S9 even after that, if it is determined that a predetermined time (of, for example, 1 hour) has elapsed since the first time power application to the heater of the heat generating unit 5 was started, the control unit 110, at step S11, stops the operation of the fan coil unit 3.

If it is determined at step S8 that the stop operation has not been performed, and if it is determined at step S9 that the predetermined time has not elapsed since the first time power application to the heater of the heat generating unit 5 was started, the control unit 110, at step S10, determines whether or not a request has been issued to switch over the temperature change patterns. If there has been issued a request for switching over the temperature change patterns at step S10, the process goes back to step S1, where the control unit 110 reads a new temperature change pattern. Thus, the fan coil unit 3 operates thereafter so as to change the room temperature in accordance with the newly-read temperature change pattern. On the other hand, if there has not been issued a request for switching over the temperature change patterns at step S10, the process goes back to step S2. Thus the fan coil unit 3 operates thereafter so as to change the room temperature in accordance with the previously-read temperature change pattern.

The fan coil unit 3 is operated as described above, whereby the human M in the room interior 1a is alternately subjected to a thermal stimulus by the hot air blown out from the fan coil unit 3 and a cold stimulus by the cool air blown out also from the fan coil unit 3. Placing the human M in the room 1 where the fan coil unit 3 is operated in this manner for 1 hour or more per day preferably for five or more consecutive days activates the physiological functions of the human M, in particular the motor functions of peripheral blood vessels. As a result, the blood pressure regulatory functions, the blood circulation regulatory functions, and the thermoregulatory functions are enhanced, thereby to achieve the effects of preventing hypertension, preventing reduction of metabolism and repair functions, accelerating recovery from fatigue, and preventing heat strokes.

1. A method for activating physiological functions characterized by alternately giving to a human body a thermal stimulus in a first temperature range of from 31 to 34° C. and a cold stimulus in a second temperature range of from 18 to 22° C.

2. The method for activating physiological functions according to claim 1, characterized in that the temperature changed from the first temperature range to the second temperature range is continuously maintained for a predetermined time within the second temperature range.

3. The method for activating physiological functions according to claim 1 or 2, characterized in that the temperature changed from the second temperature range to the first temperature range is continuously maintained for a predetermined time within the first temperature range.

4. The method for activating physiological functions according to any one of claims 1 to 3, characterized in that the thermal stimulus and the cold stimulus are alternately given to a human body for 1 hour or more per day for five or more consecutive days.

5. An apparatus for activating physiological functions characterized by comprising temperature changing means configured to give alternately to a human body a thermal stimulus in a range of from 31 to 34° C. and a cold stimulus in a range of from 18 to 22° C.

6. The apparatus for activating physiological functions according to claim 5, characterized in that the temperature changing means is a heat pump type air conditioner that selectively generates hot air and cool air.

7. The apparatus for activating physiological functions according to claim 5, characterized in that the temperature changing means includes a cold heat generating unit having a Peltier element and a heat generating unit having a Peltier element or a heater.

8. The apparatus for activating physiological functions according to claim 5, characterized in that the temperature changing means includes a cold heat generating unit having a cooling machine and a heat generating unit having a heater.
